(19) United States
(12) Patent Application Publication
(10) Pub. No.: US 2015/0117488 A1
(43) Pub. Date: Apr. 30, 2015

(54) PVT CELL FOR FOAMY OIL

(71) Applicant: Intevep, S.A., Caracas (VE)

(72) Inventor: Arturo Borges, Estado Miranda (VE)

(73) Assignee: Intevep, S.A., Caracas (VE)

(21) Appl. No.: 14/067,184

(22) Filed: Oct. 30, 2013

(51) Int. Cl. G01N 33/24; G01N 25/02

(52) U.S. Cl. CPC G01N 33/24; G01N 25/02
USPC 374/45

(57) ABSTRACT

A PVT system for evaluating foamy heavy and extra-heavy hydrocarbons, including a cell having a wall defining an inner space; a floating piston slidable in the inner space; a stirring mechanism slidable into the inner space and operative to mix a hydrocarbon sample in the inner space; a volumetric pump associated with the cell for selectively increasing and decreasing pressure on the floating piston, and communicated with the inner space to control pressure in the space; an oven for heating the cell, wherein the cell is mounted within the oven; an inversion mechanism for inverting the cell at least about 180°; and a frame supporting the oven, wherein the wall of the cell and the oven have corresponding elongated transparent sections arranged to allow visual inspection of fluid in the inner space along an entire longitudinal extent of the inner space.

### BACKGROUND OF THE INVENTION

The invention relates to a thermodynamic measurement device for evaluating properties of a fluid placed in the device. Such devices allow control of one or more of the pressure, volume and temperature of a space containing a sample of fluid such that properties of the fluid can be evaluated at different settings or values of the modified parameter.

Conventional PVT cells typically use mercury in some measurements, which can pose a health risk to the user. In addition, such cells are typically blind, meaning that there is no possibility of visually monitoring of what is happening in the cell. Further, conventional PVT cells typically take a long time to complete an experiment, and measurements produced are somewhat unreliable.

In the field of production and transportation of crude hydrocarbons such as heavy and extra-heavy hydrocarbons, hydrocarbon fluids are frequently encountered which have a tendency to foam under certain conditions. It is important to know these conditions, since the formation of foam in the reservoir near the well bore or in the production or transportation equipment, can be detrimental to the overall process. Further, a large quantity of hydrocarbons with tendency to foam are heavy or extra-heavy hydrocarbons, for example having an API gravity of less than about 10 or even 8° API.

Conventional PVT cells are not well suited for use with hydrocarbons having a tendency to form foam, nor are they well suited to heavy and extra-heavy hydrocarbons. The need exists, therefore, for a safe and accurate PVT cell which can be used to accurately evaluate the thermodynamic properties of heavy and extra-heavy hydrocarbons, particularly those with a tendency to foam. The present invention provides such a device, and method, as well as further details and characteristics which will be further discussed below.

### SUMMARY OF THE INVENTION

In accordance with the present invention, a PVT system is provided for evaluating foamy heavy and extra-heavy hydrocarbons, which system comprises a cell having a wall defining an inner space; a floating piston slidable in the inner space; a stirring mechanism slidable into the inner space and operative to mix a hydrocarbon sample in the inner space; a volumetric pump associated with the cell for selectively increasing and decreasing pressure on the floating piston, and communicated with the inner space to control pressure in the space; an oven for heating the cell, wherein the cell is mounted within the oven; an inversion mechanism for inverting the cell at least about 180°; and a frame supporting the oven, wherein the wall of the cell and the oven have corresponding elongated transparent sections arranged to allow visual inspection of fluid in the inner space along an entire longitudinal extent of the inner space.

In further accordance with the invention, a method is provided for conducting a PVT analysis of a foamy heavy or extra-heavy hydrocarbon, wherein the method comprises the steps of placing a sample of a foamy heavy or extra heavy hydrocarbon into a PVT system comprising a cell having a wall defining an inner space; a floating piston slidable in the inner space; a stirring mechanism slidable into the inner space and operative to mix a hydrocarbon sample in the inner space; a volumetric pump associated with the cell for selectively increasing and decreasing pressure on the floating piston, and communicated with the inner space to control pressure in the space; an oven for heating the cell, wherein the cell is mounted within the oven; an inversion mechanism for inverting the cell at least about 180°; and a frame supporting the oven, wherein the wall of the cell and the oven have corresponding elongated transparent sections arranged to allow visual inspection of fluid in the inner space along an entire longitudinal extent of the inner space; altering one property of pressure, volume or temperature in the inner space; mixing the sample while allowing the sample to reach equilibrium, and observing phases present in the cell through the transparent sections.

### BRIEF DESCRIPTION OF THE DRAWINGS

A detailed description of preferred embodiments of the present invention follows, with reference to the attached drawings wherein:

FIG. 1 schematically illustrates a system in accordance with the present invention;

FIG. 2 is a partially sectioned schematic view of a system in accordance with the present invention;

FIG. 3 is a partially sectioned schematic view of a system in accordance with the present invention;

FIGS. 4 and 5 show specific details of a cell component in accordance with the present invention;

FIG. 6 illustrates components of the cell of FIGS. 4 and 5;

FIG. 7 sequentially illustrates operation of the system of the present invention with a foamy fluid;

FIGS. 8a and 8b show an example of constant composition expansion process and isolation of bubble points Pb′ and Pb; and

FIG. 9 further illustrates transparent portions of components of the present invention.

### DETAILED DESCRIPTION

The invention relates to a PVT cell and system for use in evaluating heavy and extra heavy hydrocarbons, particularly such hydrocarbons which have a tendency to foam.

As discussed above, conventional equipment is not well designed to handle highly viscous heavy and extra-heavy hydrocarbons with a tendency to foam, and existing equipment is unreliable and time-consuming in order to attempt analysis of such fluids. Further, a large number of conventional systems use mercury for some measurements, and this is a hazardous substance, particularly to those using the device.

In addition, it is highly desirable to have a system which can accurately and safely produce thermodynamic analysis of heavy and extra-heavy hydrocarbon fluids, particularly those which have a tendency to foam. For example, such fluids are produced from extremely large reservoirs throughout Venezuela, and the tendency to foam can cause significant problems in production and transportation of the hydrocarbons.

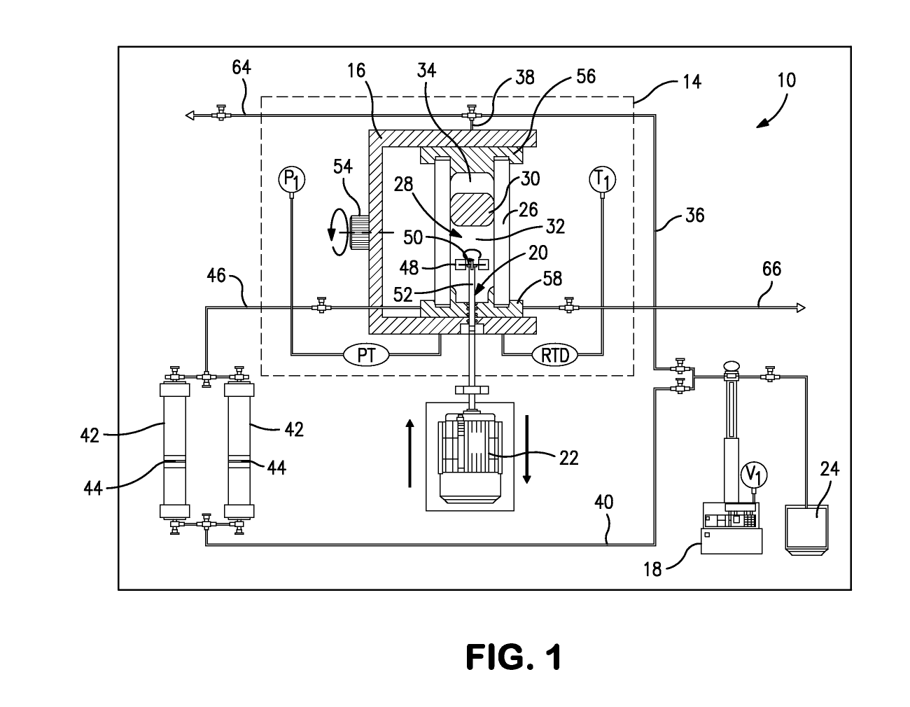

FIG. 1 illustrates a system in accordance with the present invention which is well suited to conducting thermodynamic analysis of such fluids.

FIG. 1 shows system 10 including a PVT cell 12 which is mounted in an oven schematically illustrated at 14, and supported on a frame 16 which allows a 180° inversion of cell 12. A pressure source such as a volumetric pump 18 is communicated with cell 12, preferably in two different paths to allow adjustment of pressure and volume within cell 12, as will be discussed below.

Further, a stirring mechanism 20 can be operated within cell 12 to mix fluids within cell 12, and a motor 22 can be releasably engaged with mixing structure 20 to mix fluids when desired. Pressure and temperature measurements within cell 12 are taken using pressure and temperature sensors P1, T1. Further, a control unit 24 is shown which can be provided in the form of any computing device such as a desktop or laptop computer, dedicated mainframe or the like, and can be communicated with all components and sensors of system 10 to appropriately control system 10 and record all data and measurements collected thereby.

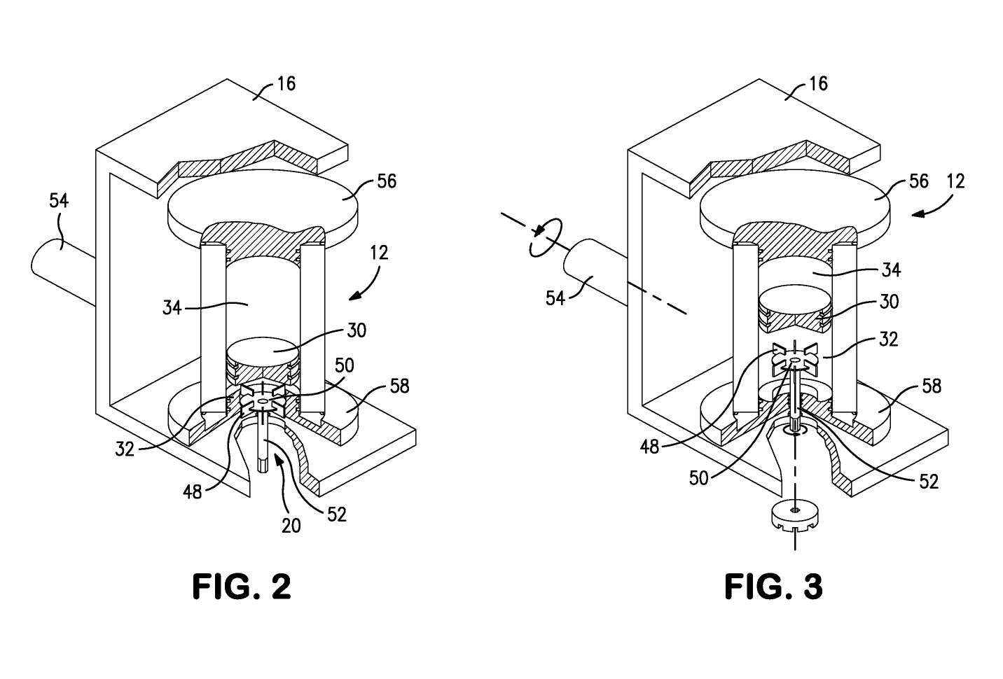

Referring also to FIGS. 2 and 3, cell 12 can preferably have a wall 26 which defines an inner space 28 for holding a fluid sample to be evaluated. Wall 26 can advantageously be formed into a cylinder or tube-shape, as shown.

A floating piston 30 is slidably positioned within inner space 28 and substantially sealingly interacts with an inner surface of wall 26. As will be evident from a consideration of FIG. 1, piston 30 divides inner space 28 into two different sections. One section 32, will typically contain the fluid to be evaluated, while the other section, 34, receives pressure from pump 18 which is used to position floating piston 30 to a desired location so as to adjust volume of section 32. Piston 30 advantageously has sufficient seals and/or engagement with the inner surface of wall 26 that leakage past piston 30 in either direction is substantially prevented.

In order to control position of piston 34, pressure can be conveyed from pump 18 through line 36 and inlet 38 which is communicated with section 34 of inner space 28. Increasing the pressure in section 34 moves piston 30 so as to decrease the volume of section 32 in which the sample is located. It is with this structure that the volume of the sample can be adjusted during thermodynamic analysis, and such thermodynamic analysis is understood by a person of ordinary skill in the art.

Pump 18 is also communicated with section 32 within cell 12 such that pressure within section 32 can be increased without moving piston 30. Pump 18 is preferably connected, in this fashion, through a line 40 which can lead to one or more piston cylinders 42 which, through pistons 44, can convey pressure through line 46 and into section 32 of cell 12 as desired.

Still referring to FIGS. 1-4 and 6, the mixing structure 20 and motor 22 will be further discussed. As shown, mixing structure 20 can be provided in the form of a plurality of vanes 48 extending from a hub 50 which is connected to a shaft 52. Shaft 52 is releasably connected to motor 22 such that, when system 10 is in the position of FIG. 1, shaft 52 can be rotated by motor 22 so as to rotate vanes 48 on hub 50 within section 32 where the sample of fluid to be analyzed is present. Vanes 48 can have any shape or structure which would be effective at mixing the heavy hydrocarbons toward which the invention is directed. These structures could include turbine or propeller type vanes, for example.

As also illustrated in FIGS. 1-3, frame 16 supporting cell 12 is advantageously mounted for at least 180° rotation or inversion so as to allow a good mixing of the sample positioned therein. FIGS. 1-3 show frame 16 mounted on a shaft 54 which can be rotated as schematically illustrated by the arrows shown in FIGS. 1 and 3 to fully invert cell 12. The motor or other structure for causing such rotation is not shown in the drawings, but would be readily known to a person skilled in the art. It should be appreciated that cell 12 configured as described can be rotated or inverted within oven 14 through rotation of frame 16 around shaft 54.

In order to rotate cell 12, it should be appreciated that shaft 52 of mixing structure 20 should first be disconnected from motor 22, and to facilitate this disconnection, motor 22 can be vertically movable relative to shaft 52 as shown by the arrows in FIG. 1.

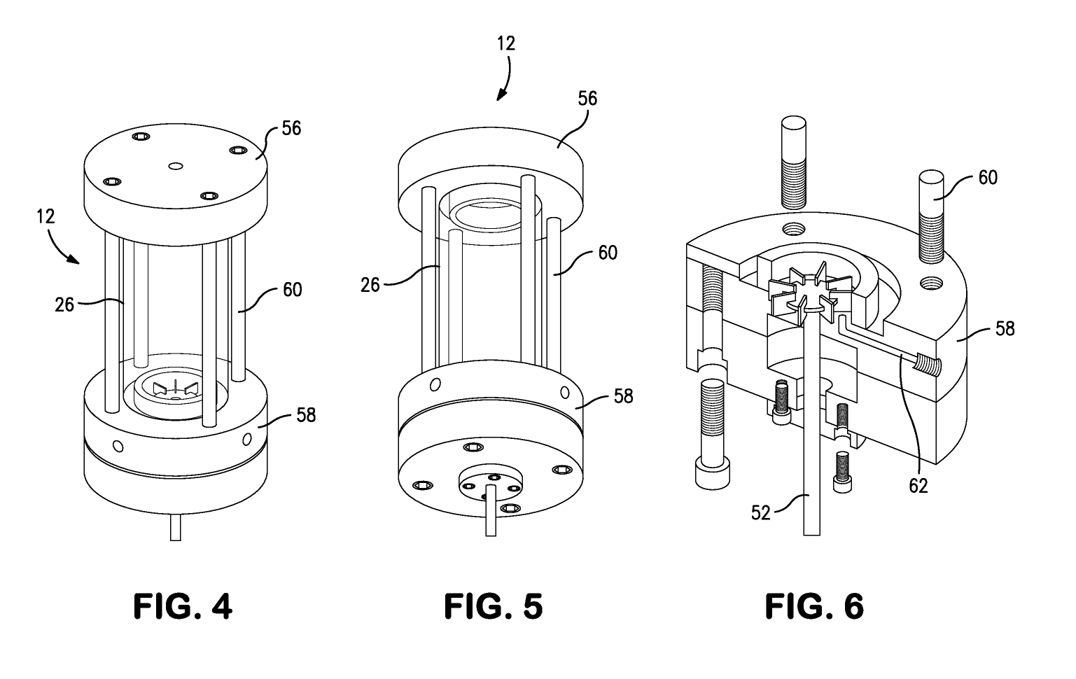

Turning now specifically to FIGS. 2 and 3, it should be appreciated that wall 26 of cell 12 can advantageously be provided as a transparent member, and this is also illustrated in FIGS. 4 and 5. This section being transparent is advantageous in accordance with the present invention as it allows observation of fluid within cell 12 as it is being analyzed, and further allows visual monitoring of foam formation within cell 12. To this end, corresponding portions of frame 16 and oven 14 should also be transparent, and preferably should have at least transparent sections which extend the entire vertical height of cell 12 so that foam can be observed at either end of cell 12 or any position therebetween.

It should be appreciated that either end of cell 12 is closed by an end cap 56, 58, and that some flow lines and structures must past through said end caps to allow for proper functioning of the device.

For example, end cap 56 which is opposite to mixing structure 20 is a substantially solid end cap but for being provided with flow channels (not shown) for conveying pressure from pump 18 through lines 36, 38 and into pressurized section 34 of inner space 28.

End cap 58 at the same side of mixing structure 20 is slightly more complex, as shaft 52 of mixing structure 20 must sealingly pass through end cap 58 as shown in FIGS. 2, 3 and 6.

As shown in FIGS. 4 and 5, end caps 56, 58 can extend laterally beyond the perimeter of cell 12, and this advantageously allows a plurality of rods 60 or other support structures to be connected between end caps 56, 58 and thereby provide additional strength and durability to cell 12. FIG. 6 further illustrates end cap 58 and shows a flow passage 62 which passes from a perimeter of end cap 58 inwardly to a surface which communicates with section 32. Flow passage 62 is advantageously communicated with pressure from pump 18, preferably conveyed through piston cylinders 42 and line 46, and this structure is used to adjust pressure within section 32 as desired.

Returning to FIG. 1, it should be appreciated that an outlet line 64 is connected to line 36 as shown and can be used to vent pressure from section 34 of cell 12 or allow release of pressure directly from pump 18 as desired. In addition, an outlet line 66 can also be provided, preferably from end cap 58, and this line can be used to bleed released gas from within section 32 as desired. FIG. 1 shows a number of valves along the various lines which can be used to control the different flows discussed, and these valves, it should be appreciated, would preferably be under control of controller 24 in order to produce a fully automated system, preferably which provides all data to an operator in digital form.

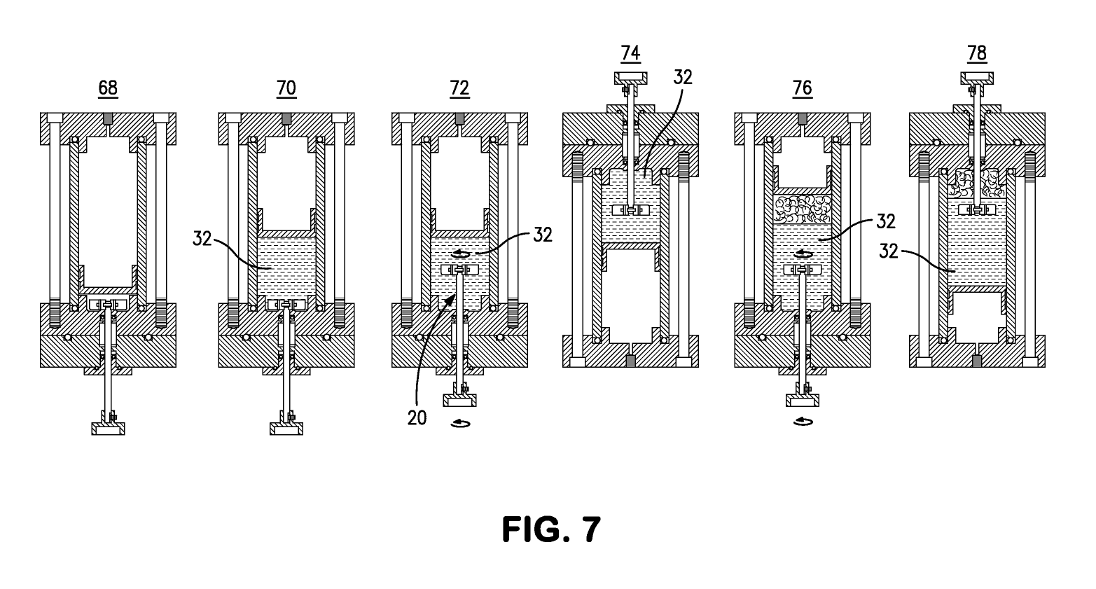

Turning now to FIG. 7, a first view 68 shows cell 12 in accordance with the present invention in an empty condition. View 70 shows cell 12 containing a sample of fluid in section 32. View 72 shows mixing structure 20 being used to stir the sample fluid within section 32 until a stabilization point is reached. View 74 shows cell 12 in an inverted position. View 76 illustrates cell 12 which now contains two phases, namely a liquid and foam phase, along with stirring through mixing structure 20 such that the two phases are stabilized.

View 78 shows cell 12 with two phases in an inverted position.

Views 72 and 74 of FIG. 7 show how to obtain a good equilibrium with the device of the present invention by inverting the cell in order to find a best homogenization for each pressure value (one phase region in this case) and analogically in views 76 and 78 for two phases regimes.

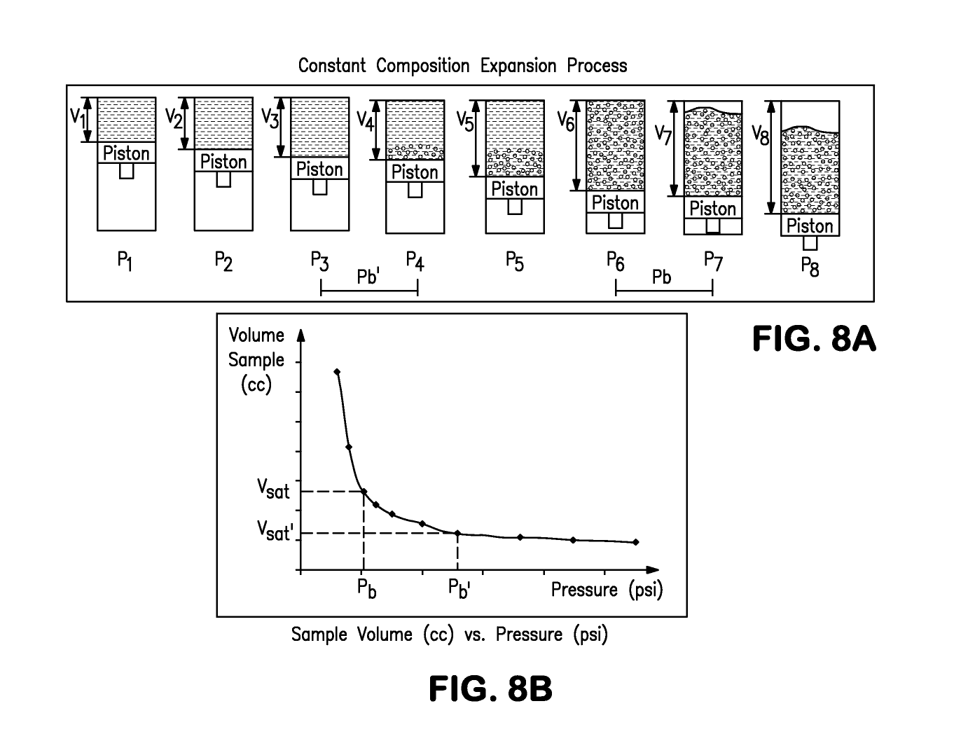

FIGS. 8a and 8b show an experimental constant composition expansion process at reservoir temperature using an extra heavy fluid with a foamy behavior. The different views provided are conditions observed at different equilibriums (after stirring and stabilizing at each point). Pressures P₁ through P₈ with corresponding sample volumes (V₁ through V₈) are shown. When plotting sample volume vs. pressure, it is possible to determine the pseudo bubble point (P_{b}′), in this case between P₃ and P₄, when foam starts to be produced, and the bubble point (P_{b}), in this case between P₆ and P₇, when free gas starts to be produced in a free gas cap in the top of the cell. The device of the present invention can be used to evaluate the foamy heavy hydrocarbon behavior during the constant composition expansion process.

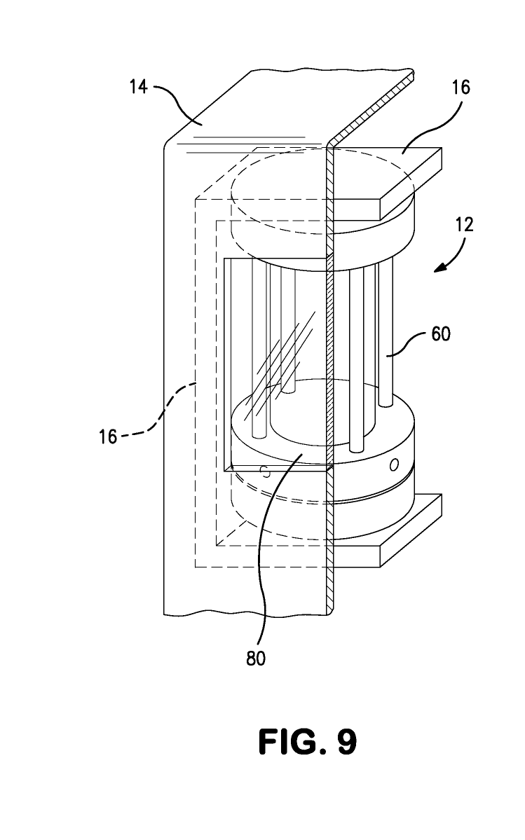

FIG. 9 shows an arrangement with cell 12 within oven 14 and showing a transparent portion 80 of a wall of oven 14 to allow visual inspection of cell 12 therein. It should be appreciated that frame 16, in order to allow reliable mounting and rotation within oven 14, may have wall sections which extend in ways that would obstruct view of cell 12. Thus, in one aspect of the invention, the frame 16 may also have transparent sections for example aligned within oven 14 behind section 80, or could be made having slots or other clear areas to allow good viewing of cell 12 within oven 14 as desired, that is, without opaque sections between section 80 and cell 12. It should be appreciated that the support rods 60 of cell 12 allow for the cell to have transparent walls to provide complete visibility into same, while preserving structural strength of cell 12.

The above features of cell 12, oven 14 and frame 16 combine to provide a system along with all control equipment, which can produce excellent experimental precision when foamy heavy hydrocarbon phases are studied, because the pseudo bubble point (P_{b}′) and the bubble point (P_{b}) can both be identified with precision. These parameters are very important to evaluate as they are highly relevant production mechanisms in reservoir studies. Also, this information allows identification of the volumetric behavior, density and compressibility of foamy phase during depletion studies at reservoir condition.

It should be appreciated that the present disclosure has been given in terms of a preferred embodiment. The scope of the invention is not to be viewed as being limited by this embodiment, but rather as being defined by the scope of the appended claims.

1. A PVT system for evaluating foamy heavy and extra-heavy hydrocarbons, comprising:
a cell having a wall defining an inner space;
a floating piston slidable in the inner space;
a stirring mechanism slidable into the inner space and operative to mix a hydrocarbon sample in the inner space;
a volumetric pump associated with the cell for selectively increasing and decreasing pressure on the floating piston, and communicated with the inner space to control pressure in the inner space;
an oven for heating the cell, wherein the cell is mounted within the oven;
an inversion mechanism for inverting the cell at least about 180°; and
a frame supporting the oven, wherein the wall of the cell and the oven have corresponding elongated transparent sections arranged to allow visual inspection of fluid in the inner space along an entire longitudinal extent of the inner space.

2. The system of claim 1, further comprising a motor for driving the stirring mechanism, the motor being selectively connectable and disconnectable to the stirring mechanism to allow disconnect of the motor from the stirring mechanism when the cell is to be inverted.

3. The system of claim 1, wherein the floating piston is moveable within the inner space by increasing and decreasing pressure applied to the floating piston by the volumetric pump.

4. The system of claim 1, wherein the wall of the cell is entirely transparent.

5. The system of claim 4, further comprising end caps mounted at each end of the wall of the cell, and a plurality of support rods extending parallel to the wall to support the end caps relative to each other.

6. The system of claim 5, wherein the support rods are outside of the inner space.

7. The system of claim 1, wherein the stirring mechanism comprises stirring vanes mounted on a rod which extends into the inner space through one end cap of the cell.

8. The system of claim 1, further comprising piston cylinders communicated with the inner space and the volumetric pump for controlling pressure in the inner space.

9. The system of claim 1, wherein the frame is arranged to have no obstructing structures between the transparent sections of the cell and the oven.

10. The system of claim 1, wherein the frame has a transparent section positioned between the transparent sections of the cell and the oven.

11. A method for conducting a PVT analysis of a foamy heavy or extra heavy hydrocarbon, comprising the steps of:
placing a sample of a foamy heavy or extra heavy hydrocarbon into a PVT system comprising
a cell having a wall defining an inner space;
a floating piston slidable in the inner space;
a stirring mechanism slidable into the inner space and operative to mix a hydrocarbon sample in the inner space;
a volumetric pump associated with the cell for selectively increasing and decreasing pressure on the floating piston, and communicated with the inner space to control pressure in the inner space;
an oven for heating the cell, wherein the cell is mounted within the oven;
an inversion mechanism for inverting the cell at least about 180°; and
a frame supporting the oven, wherein the wall of the cell, and the oven have corresponding elongated transparent sections arranged to allow visual inspection of fluid in the inner space along an entire longitudinal extent of the inner space;
altering one property of pressure, volume or temperature in the inner space;
mixing the sample while allowing the sample to reach equilibrium; and
observing phases present in the cell through the transparent sections.

12. The method of claim 11, further comprising carrying out the mixing and observing steps at different values of the property applied in the altering step to predict foaming characteristics of the sample.

13. The method of claim 11, wherein the frame is arranged to have no obstructing structures between the transparent sections of the cell and the oven.

14. The method of claim 11, wherein the frame has a transparent section positioned between the transparent sections of the cell and the oven.
